(19) United States
(12) Patent Application Publication
(10) Pub. No.: US 2024/0218319 A1
(43) Pub. Date: Jul. 4, 2024

(54) METHOD FOR PRODUCING CELL GROWTH MEDIUM

(71) Applicant: Solar Foods Oy, Lappeenranta (FI)

(72) Inventors: Dilek ERCILI-CURA, Lappeenranta (FI); Pasi VAINIKKA, Lappeenranta (FI); Juha-Pekka PITKÄNEN, Lappeenranta (FI); Sissel RØNNING, ÅS (NO); Sileshi WUBSHET, ÅS (NO); Nina SOLBERG, ÅS (NO); Lene ØVERBY, ÅS (NO); Katinka DANKEL, ÅS (NO)

(73) Assignee: Solar Foods Oy, Lappeenranta (FI)

(21) Appl. No.: 18/553,904

(22) PCT Filed: Apr. 22, 2022
(86) PCT No.: PCT/FI2022/050264

§ 371 Date: Oct. 4, 2023

(30) Foreign Application Priority Data
Apr. 28, 2021 (FI) 20215493

(51) Int. Cl. C12N 5/00; C12M 1/00; C12N 1/20

(52) U.S. Cl. CPC C12N 5/0018; C12M 47/02; C12M 47/08; C12M 47/14; C12M 47/20; C12N 1/20

(57) ABSTRACT

A method of producing a cell growth medium. The method includes cultivating microbial cells by gas fermentation to obtain a biomass slurry, concentrating the biomass slurry by separating and removing a liquid phase from a solid phase, homogenizing the concentrated biomass slurry with high pressure homogenization, wherein the microbial cells are at least partially degrading, treating the homogenized biomass slurry by adding at least one protease, and heating the treated biomass slurry.

Reference is made to the Identification of the Microorganism, with the identification reference given by the DEPOSITOR of SoF1, having the Accession number given by the INTERNATIONAL DEPOSITORY AUTHORITY of VTT E-193585, received on Jun. 11, 2019, (date of original deposit).

### TECHNICAL FIELD

The present disclosure relates generally to cell growth medium or cell growth mediums; and more specifically to methods of producing cell growth mediums. Moreover, the present disclosure also relates to systems for producing cell growth mediums.

### BACKGROUND

Humans depend on a variety of food sources to meet their requirements of a balanced diet. Typically, a balanced human diet comprises protein, carbohydrates, fats, vitamins and minerals in proper proportions. While animal-based food sources provide most of the aforementioned nutrients, they are not a sustainable solution since wide acres of land, food and water resources are expensed for rearing animals. Moreover, the reared animal is sacrificed in return for only a little amount of useful products (such as animal meat, serum, and the like), thus forming an unethical aspect of animal husbandry. Besides, meat industry is a leading producer of atmospheric carbon dioxide (CO₂), globally. Therefore, there is a long-felt need for a sustainable animal protein production, more specifically, cell-cultured meat, using a suitable cell growth medium. Notably, the most significant cost driver for efficient culturing of cells and resulting products intended for human consumption is the growth medium (comprising cell growth medium and growth supplements), which can account for up to 96% of the total cost. Notably, cell growth medium and growth supplements are the limiting factor for sustainable large-scale culturing of cells and resulting products.

Traditional growth medium contains commercially available blood serum that is harvested from bovine foetuses at the slaughterhouse. Besides, this solution does not exclude ethical and environmental concerns relevant to animal husbandry. Moreover, such commercially available serum supplements are expensive and not necessarily food-grade. Therefore, identifying inexpensive food-grade alternatives to serum supplements is crucial. However, one of the technical challenges is the formulation of a sustainable, serum-free cell culture medium. Another challenge is to provide a cell growth medium, which contains sufficient amount of iron and vitamin B12, which are normally not present in plant based growth mediums.

Recent advances in the cell culture technology has paved way for identifying bioactive peptides derived from hydrolysis of food proteins as potential compounds for application as cell-promoting agents (Roeseler et al., 2017). Moreover, it has been previously demonstrated that supplementation of fibrous proteins and glycosaminoglycans improved the cell growth of primary skeletal bovine muscle cells (Rønning et al., 2013), and bioactive peptides from chicken by-products (Lima et al., 2019) improved glucose uptake. Furthermore, eggshell membrane is another promising fibrous material with anti-inflammatory properties that contains active components (carbohydrates, proteins, and peptides), growth factors and enzymes supporting cell growth and survival (Ahmed et al., 2017; Vuong et al., 2017). Additionally, Nofima has shown that hydrolysates from different by-products, including chicken carcass, cod backbone, eggshell membrane, egg white powder, pork plasma and yeast extract all have the potential of being included in a tailor-made serum-free medium (Andreassen et al., 2020).

During a transition phase, by-products from food industry can be used as ingredients in sustainable, serum-free growth media. Moreover, bioactive peptides derived from hydrolysis of food proteins, e.g. plant proteins, have been shown to be potential compounds for application as cell-promoting agents (Roeasler et al., 2017). One problem related to hydrolysates, which are commercially available, is that keeping their composition standardized is challenging. Therefore, the long-term goal for culturing meat is to use protein sources entirely free from agriculture.

Therefore, in light of the foregoing discussion, there exists a need to overcome drawbacks associated with conventional cell growth mediums that are affordable, contain only food-grade components, and effective to promote cell growth and differentiation.

### SUMMARY

The present disclosure seeks to provide a method of producing a cell growth medium. The present disclosure also seeks to provide a system for producing a cell growth medium. The present disclosure seeks to provide a solution to the existing problem of employing animal-based or alternative plant-based cell growth mediums for cell cultured meat production. An aim of the present disclosure is to provide a solution that overcomes at least partially the problems encountered in prior art.

In one aspect, an embodiment of the present disclosure provides a method of producing a cell growth medium, the method comprising
cultivating microbial cells by gas fermentation to obtain a biomass slurry;
concentrating the biomass slurry by separating and removing a liquid phase from a solid phase;
homogenizing the concentrated biomass slurry with high pressure homogenization, wherein the microbial cells are at least partially degrading;
treating the homogenized biomass slurry by adding at least one protease; and
heating the treated biomass slurry.

In another aspect, an embodiment of the present disclosure provides a system for producing a cell growth medium, the system comprising:
a bioreactor for cultivating microbial cells by gas fermentation to obtain a biomass slurry;
a separator for separating a solid phase and a liquid phase of the biomass;
a homogenizing device for homogenizing the biomass slurry with high pressure homogenization for at least partially degrading the microbial cells;
a reaction chamber for treating the biomass slurry by adding at least one protease; and
an incubator for heating the biomass slurry.

Embodiments of the present disclosure substantially eliminate or at least partially address the aforementioned problems in the prior art, and provides an efficient method of producing the cell growth medium derived from microbial cells. Beneficially, the aforementioned cell growth medium is a sustainable alternative to fetal bovine serum (FBS) used in cell culturing. Additionally, beneficially, the aforementioned cell growth medium is a protein-rich powder or slurry generated through a fermentation-like process where living microbes use electricity, air and water to produce protein. Besides proteins, the aforementioned cell growth medium naturally comprises iron, B-vitamins, other relevant micronutrients and nucleic acids in substantial amounts which of all serve as growth promoting factors. Moreover, the aforementioned cell growth medium is entirely free from agriculture and can be used as an ingredient for culturing meat.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a flowchart depicting steps of a method of producing a cell growth medium, in accordance with an embodiment of the present disclosure; and

FIG. 2 is a block diagram of a cell growth medium production line, in accordance with different embodiments of the present disclosure;

FIG. 3 represents graphs depicting muscle cells cultivated with the cell growth medium, showing cell metabolic activity;

FIG. 4 represents graphs depicting DNA normalised to control cells.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, an embodiment of the present disclosure provides a method of producing a cell growth medium, the method comprising
cultivating microbial cells by gas fermentation to obtain a biomass slurry;
concentrating the biomass slurry by separating and removing a liquid phase from a solid phase;
homogenizing the concentrated biomass slurry with high pressure homogenization, wherein the microbial cells are at least partially degrading;
treating the homogenized biomass slurry by adding at least one protease; and
heating the biomass slurry.

In another aspect, an embodiment of the present disclosure provides a system for producing a cell growth medium, the system comprising:
a bioreactor for cultivating microbial cells by gas fermentation to obtain a biomass slurry;
a separator for separating a solid phase and a liquid phase of the biomass;
a homogenizing device for homogenizing the biomass slurry with high pressure homogenization for at least partially degrading the microbial cells;
a reaction chamber for treating the biomass slurry by adding at least one protease; and
an incubator for heating the biomass slurry.

The present disclosure provides the aforementioned method of producing the cell growth medium. Moreover, the produced is a sustainable cell growth medium whose production is completely delinked from agriculture and animal husbandry. The said cell growth medium is typically a single cell protein powder generated through a fermentation-like process, in which the proprietary microbe uses electricity, air and water to grow and produce proteins. Beneficially, the final microbial biomass contains more than 60% protein in dry matter which can be hydrolyzed into peptides using food-grade proteases. Additionally, beneficially, one advantage of the said cell growth medium when compared to other hydrolysates, especially plant-based hydrolysates, is that production thereof is highly controlled and could be automated to produce same peptides and amino acids during each hydrolysis step. Moreover, said cell growth medium naturally comprises iron, B-vitamins, and other relevant micronutrients and nucleic acids in substantial amounts which of all serve as growth promoting factors.

It will be appreciated that the cell growth medium obtained from the aforesaid method is a more sustainable, healthier and cruelty-free alternative to standard animal-based serum (such as fetal bovine serum) obtained after sacrificing animals. Moreover, such cell growth medium may be suitable for use by a wide demographic of consumers identified as vegetarians or vegans. Furthermore, the production process of said cell growth medium contributes negligibly to the global warming effect as compared to the production of animal-based serum that releases large amounts of carbon dioxide in the environment.

Throughout the present disclosure, the term “cell growth medium” as used herein refers to a defined recipe as a source of several nutrients and growth factors essential for cell growth. Optionally, cell growth medium could be added to a cell culture media as an ingredient thereof. Specifically, cell growth medium is a single cell protein powder derived from microbial biomass. Beneficially, the cell growth medium is an animal-free and plant-free product. Additionally, beneficially, the microbial cell growth medium is suitable for mostly all types of cell cultures. Growth mediums typically comprise various amino acids, vitamins, inorganic minerals, lipids, nucleic acid derivatives, hormones (such as insulin, adrenocortical hormone, steroids, and so forth), growth factors (such as epidermal growth factors, fibroblast growth factors, and so forth), cell adherence factors (such as laminin, fibronectin, and so forth), salts, carbohydrates, antibiotics, and so on.

Optionally, the microbial cells comprise an isolated bacterial strain VTT-E-193585 or a derivative thereof. The said isolated bacterial strain or a derivative thereof is typically a Gram-negative bacterium (which do not retain crystal violet stain used in the gram-staining method). It will be appreciated that the said isolated bacterial strain or a derivative thereof is genetically stable and can be grown in a broad range of process conditions, ranging from optimal to stressful conditions, over time. The term “genetically stable” as used herein, refers to a characteristic of a species or a strain/isolate to resist changes and maintain its genotype over multiple generations or cell divisions, ideally hundreds to thousands. Optionally, the said isolated bacterial strain or a derivative thereof utilize hydrogen gas as energy source and carbon dioxide as carbon source. Beneficially, the said strain or the derivative thereof comprises proteins, iron and vitamin B12. Using the microbial cells comprising isolated bacterial strain VTT-E-193585 or a derivative thereof in the present method enables to obtain a cell growth medium, which is rich in proteins, iron and vitamin B12 and therefore there is no further treatment necessary for adding said components to the cell growth medium for animal protein production.

The method comprises cultivating microbial cells by gas fermentation to obtain a biomass slurry. The microbial cells are normally cultivated in a special vessel, referred to as a bioreactor. Optionally, a small amount of microbial cells (namely, inoculum) is loaded into the bioreactor from a stock solution, and cultivated in controlled conditions. The bioreactor is typically supplied with air, water, nutrients, growth supplements, light, optimum temperature conditions, and so forth, suitable for growth of the microbial cells. In this regard, microbial cells use one or more nutrients and growth supplements in the cell growth medium as a carbon source, a nitrogen source, and an energy source to produce a high-cell density biomass. Notably, besides the bioreactor, microbial cells could be grown through any conventional process know to a person skilled in the art.

The term “biomass” as used herein refers to a measure of amount of living component (namely, bacteria) in a sample. Notably, the biomass typically comprises the solid phase (namely, bacterial cells) mixed with a liquid phase (such as water), thereby, resulting in a viscous biomass slurry. Optionally, the solid phase of the biomass slurry may include proteins, carbohydrates, fats, minerals (such as calcium, iron), vitamins, fibre and the like. Optionally, the biomass could be produced by a continuous cultivation or a batch cultivation. It will be appreciated that microbes have shorter reproduction time and, thus, can be grown rapidly to produce a high-cell density biomass that is then harvested for different applications thereof, such as food ingredient powder, for example.

The term “gas fermentation” as used herein refers to a process of employing gases like hydrogen, carbon dioxide and carbon monoxide as energy and carbon sources by the microbial cells for growth thereof. Optionally, a feed for cultivating by gas fermentation comprises at least one of selected from CO₂, CH₄, H₂, O₂, NH₃, minerals. Moreover, addition of NH₃ provides a nitrogen source for the bacterial cells. It will be appreciated that addition of minerals, such as minerals containing ammonium, phosphate, potassium, sodium, vanadium, iron, sulphate, magnesium, calcium, molybdenum, manganese, boron, zinc, cobalt, selenium, iodine, copper and/or nickel enhance growth of bacterial cells. Optionally, said minerals comprises less than 1 g/L of chloride salts, such as less than 0.25 g/L of chloride salts, e.g. less than 0.1 g/l of chloride salts, preferably no chloride salts.

Moreover, the biomass slurry is concentrated by separating and removing the liquid phase from the solid phase. It will be appreciated that concentrating the biomass slurry refers to removing excess liquid phase from solid phase of the biomass slurry. Notably, the liquid phase of the biomass comprises hydrolysed components of the cell wall structures including the LPS-containing endotoxins. Separating and removing liquid phase from the solid phase leaves the concentrated biomass with reduced endotoxins therein. Optionally, separating is carried out with a separation method selected from at least one of a filtration, a centrifugation. The filtration technique typically separates the liquid and solid phases through a semi-permeable membrane that allows the liquid phase to pass therethrough while retaining the solid phase over the said semi-permeable membrane. Centrifugation is typically a technique for the separation of particles according to their size, shape, density, viscosity or speed of rotor employed for separation. The centrifugation normally separates about 90-95% of liquid phase from the solid phase. At this stage, the biomass is concentrated by removal of the liquid phase, and comprising about 5-30% of dry matter in comparison to the earlier 0.5-2% dry matter, before separation.

The method further comprises homogenizing the concentrated microbial biomass slurry with high pressure homogenization, wherein the microbial cells are at least partially degrading. The term “homogenizing” as used herein refers to a means of physical disruption of microbial cell walls. Typically, homogenizing techniques exploit fluid flow, particle-particle interaction, and pressure drop to facilitate cell disruption. It will be appreciated that incubating the biomass slurry with heat treatment partially disrupts the outer membrane of cell walls, and homogenizing the biomass slurry further disrupts the cell walls. It will be appreciated that due to partial cell wall hydrolysis, proteins are partially released from the microbial cell and into the remaining liquid phase of the biomass (i.e. after the separation process). At this stage, homogenization enhances protein release from the cell interior such that the proteins are more available for enzymatic hydrolysis thereof. Homogenization will enhance protein release so that proteins have better availability for further treatment steps. For example, partial cell lyse leads to protein release from the cell interior and the cell interior becomes accessible for enzyme hydrolysis in the next stages, which will make treatment with proteases more efficient. Furthermore, during homogenization process, endotoxins are removed further resulting in a final product with 10-1000 times lower endotoxin response.

Most commonly used technique for microbial cell wall disruption include high-pressure homogenization (HPH). The term “high-pressure homogenization” as used herein refers to a physical or mechanical process of forcing a stream of sample, such as the biomass that comprises solid-phase and liquid phase (remaining post separation process), through a system, implemented as a homogenizing device (discussed in details later) that subjects the sample to a plurality of forces, such as high pressure or any combination of shear forces for example, which intend to homogenize the sample and/or reduce the particle size of any components within the sample.

Optionally, the high pressure homogenization is carried out at pressure from 900 bars up to 2000 bars. Optionally, homogenization pressure may typically be from 900, 1000, 1100, 1200, 1300, 1400, 1500, 1600, 1700, 1800 or 1900 bars up to 1000, 1100, 1200, 1300, 1400, 1500, 1600, 1700, 1800, 1900 or 2000 bars. Optionally, the high pressure homogenization is performed once, twice or thrice to result in desired homogenization. Beneficially, the homogenization process results in partial lysis of cell and increasing soluble protein content of the biomass.

The homogenized biomass slurry is treated by adding at least one protease. The term “treating” as used herein refers specifically to an enzymatic treatment of the concentrated biomass slurry, and more specifically, to enzymatic hydrolysis. The enzymatic hydrolysis refers to a process in which enzymes facilitate cleavage of bonds in larger molecules (such as of protein molecules) by addition of water to generate smaller peptides and amino acids. Optionally, treating the biomass slurry by adding at least one protease is carried out at temperature from 30° C. up to 75° C. The temperature for treating biomass slurry may typically be from 30, 35, 40, 45, 50, 55, 60, 65 or 70° C. up to 35, 40, 45, 50, 55, 60, 65, 70 or 75° C. The duration of enzymatic hydrolysis of the treating can last from 0.5 hours up to 16 hours. The duration may typically last from 0.5, 1, 2, 3, 5, 10 or 15 hours up to 1, 2, 3, 5, 10, 15 or 16 hours. In an example, the biomass slurry is treated at a temperature of 50° C. for a time duration ranging from 1-12 hours at a pH of 7.0.

Hydrolysis is normally achieved by employing at least one protease. Optionally, the at least one protease is a food-grade protease. The proteases typically uses water molecule to break down peptide bonds within protein molecules. It will be appreciated that proteolytic hydrolysis significantly improves functional (such as taste, mouthfeel) and structural (such as solubility, digestibility, protein dispensability) characteristics of proteins. Beneficially, proteolytic hydrolysis of biomass slurry enhances the biomass slurry's growth-promoting properties for bovine primary skeletal muscle cells.

Optionally, the at least one protease is selected to be at least one of a flavorzyme, an alcalase, a neutrase, a papain, a trypsin, a pepsin. Alcalase is an endopeptidase and flavorzyme is a mixture of endo- and exopeptidases. The endopeptidases typically cleaves the polypeptides within the polypeptide chain rather than at the terminal amino acids located at the ends of polypeptides. It will be appreciated that the biomass slurry in its crude form is insoluble, however, enzymatic hydrolysis with both alcalase and flavorzyme produced water-soluble fractions thereof containing proteins, peptides and amino acids. Both alcalase hydrolysates (protein hydrolysates obtained using alcalase) and flavorzyme hydrolysates (protein hydrolysates obtained using flavorzyme) are beneficial for cell cultures. When supplemented to cell cultures, both alcalase hydrolysates and flavorzyme hydrolysates improves cell viability, cell proliferation and cell number. Alcalase, being an endopeptidase, gives a hydrolysate mixture with a different molecular weight distribution than Flavourzyme, which is a mixture of endo- and exopeptidases. Hydrolysates produced using Flavourzyme contain larger fractions of small peptides and free amino acids compared to Alcalase digestion of the same raw material. Hydrolysates produced using Alcalase contains higher fraction of larger peptides. Using a combination of these two enzymes produced hydrolysates with higher proportion of small peptides. The highest yield and lowest average molecular weight was achieved by using a combination of Alcalase and Flavourzyme. Although various proteases could be used, hydrolysis of the protein slurry with flavorzyme and alcalase, used alone or in combination results in a material with higher amounts of soluble protein, amino acid and peptide content. When alcalase and/or flavorzyme was/were supplemented into the growth media of bovine skeletal muscle cell, hydrolysates of the protein slurry improved cell viability, cell proliferation and cell number more efficiently. When combining alcalase and flavourzyme together, synergy was shown by obtaining better yield and more favorable molecular weight (MW) distribution for the hydrolysates. Optionally, flavorzyme and alcalase could be used alone or in combination.

Moreover, neutrase is a broad-spectrum endopeptidase, that provides a mild hydrolysis. Optionally, neutrase could be used in isolation or in combination with an exopeptidase for superior flavor benefits. Papain shows both exo- and endopeptidase activity. Trypsin is an endopeptidase. Pepsin is an acidic endopeptidase normally active in a pH ranging from 1.5-2.5. Due to at least partially degrading the microbial cells with high pressure homogenization, the cell viability, cell proliferation and cell number increase is higher, when compared to hydrolysation without homogenizing step.

It will be appreciated that during culturing of the cells, removing blood serum completely is a dramatic and harsh condition for the cells. Although, none of the hydrolysates (animal-based or plant-based) are able to restore cell growth completely in the absence of serum (namely, fetal bovine serum) A highest concentration of 0.01 mg/ml of the biomass slurry in its crude extract form, i.e., non-hydrolyzed and 0.1 mg/ml of the protein hydrolysate using flavorzyme and/or alcalase are able to significantly improve the cell growth by 2-fold compared to no serum control. Moreover, it will be appreciated that high concentrations of biomass slurry crude extract and protein hydrolysates could lead to bacterial infection in cell cultures. Therefore, it is crucial to determine the concentrations of biomass slurry crude extract and protein hydrolysates when supplementing to cell cultures.

Moreover, optionally, the method comprises analysis of molecular weight distribution of protein hydrolysates, replacement of serum in cell cultures at varying concentrations, typically ranging from 0.0001-0.1 mg/mL, for crude biomass slurry and/or protein hydrolysates, and analysis of cell cytotoxicity, cell viability, cell proliferation and cell morphology. It will be appreciated that proteolytic hydrolysis using flavorzyme and alcalase, alone or in combination, result in a protein hydrolysate (a product of hydrolysis) with high amounts of soluble protein, amino acid and peptide content. When supplemented into the growth media of bovine skeletal muscle cell, protein hydrolysates improved cell viability, cell proliferation and cell number (concentration 0.0001-0.1 mg/mL). Moreover, from the molecular weight distribution profiles of enzymatic hydrolysis with commercial proteases (i.e., alcalase and flavorzyme), flavorzyme hydrolysate had a lower molecular weight than the alcalase hydrolysate. Furthermore, said molecular weight profile is consistent with the enzymes' different modes of action.

Optionally, the method further comprises adjusting the pH of the biomass slurry to be from 4.0 up to 10 before treating the biomass slurry by adding at least one protease. The pH of the biomass slurry could be in a range from 4, 5, 6, 7, 8 or 9 up to 5, 6, 7, 8, 9 or 10. In this regard, conventional pH adjustors (acids or bases) could be added to the biomass slurry to adjust the pH thereof. It will be appreciated that an optimum pH of the biomass slurry helps keeping active the at least one protease for treating the biomass slurry.

Moreover, the method comprises heating the treated biomass slurry. The treated biomass slurry is heated at optimum temperatures to enable enzyme inactivation and sterilization (or pasteurization) of the biomass slurry. It will be appreciated that the heating is carried out at temperatures safe for the activation of proteins and other desired nutrients in the biomass slurry. Optionally, heating the biomass slurry is carried out at temperature from 80° C. up to 130° C. The heating temperature may typically be from 80, 90, 100 or 110° C. up to 90, 100, 110, 120 or 130° C. Duration of the heating can be from 0.5 minutes up to 60 minutes. The heating duration may typically be from 0.5, 1, 2, 3, 5, 10, 15, 20, 25, 30 or 45 minutes up to 1, 2, 3, 5, 10, 15, 20, 25, 30, 45 or 60 minutes. Notably, the said heating temperature is sufficient to kill the bacterial pathogens.

Optionally, the method further comprises separating and removing insoluble components from the heated biomass slurry. The target of separating is to remove insoluble components from the soluble components of the heated biomass slurry. The soluble components may for example comprise proteins, peptides, amino acids, vitamins and minerals. The insoluble components may include for example cell debris, Separation can be done for example by centrifugal separation, membrane filtration. The supernatant from centrifugation and the permeate from filtration comprise soluble components, which can be used as the growth media.

Optionally, the method further comprises incubating the cultivated biomass slurry with a heat treatment. The term “incubating” as used herein refers to heat treatment performed at a selected temperature for a selected time. Incubating the cultivated biomass slurry reduces the amount of potential antinutrients such as lipopolysaccharides and nucleic acids. Moreover, incubating with heat treatment result in partial hydrolysis of cell wall of the microbial cells. Furthermore, partial hydrolysis of outer membrane of cell wall leads to cell interior to become more accessible, as well as partial removal of endotoxins along with the cell wall. Optionally, incubating the cultivated biomass slurry with a heat treatment is carried out at temperature ranging from 55° C. up to 80° C. for 10 up to 60 minutes. The temperature during incubation may for example be from 55, 56, 57, 58, 59, 60, 65, 70 or 75° C. up to 56, 57, 58, 59, 60, 65, 70, 75 or 80° C. The incubation period may for example be from 10, 15, 20, 25, 30, 35 or 40 minutes up to 20, 25, 30, 35, 40, 55 or 60 minutes.

Optionally, the method further comprises drying the heated biomass slurry by spray drying. The term “drying” as used herein refers to a process of drying out liquids from raw materials, such as biomass slurry. The spray drying process utilizes a spray of hot gases (such as nitrogen or oxygen) to rapidly dry the biomass. Spray drying is suitable for drying thermally-sensitive samples, such as food and pharmaceutical products. The dried biomass contains reduced levels of endotoxins after the incubation, separation and homogenization steps as compared to the samples that are not incubated, not separated and/or not homogenized. Alternatively, drying could be accomplished by using relatively low temperatures over rotating the biomass in a closed system, such as a drying drum, for example.

It will be appreciated that drying biomass slurry enables easy and effective storage and transport of the dried biomass derived therefrom. Moreover, drying the biomass slurry prevents the dried biomass from a potential infestation and thereby unfit for consumption by humans or animals.

Optionally, the method further comprises drying the concentrated biomass slurry to obtain a protein powder. The drying can be done for example by drum drying or by spray drying. As noted above, the dried biomass or biomass powder could be stored or transported for a further application thereof, such as for cell culture, for example. However, some application of the protein powder may still require mixing the protein powder with water. In this regard, 5-20% by weight of the protein powder is mixed in 80-95% by weight of sterile water until a homogenous mixture is achieved. Optionally, the protein powder is mixed with water using high shear mixing. It will be appreciated that high shear mixing ensures complete hydration and breaking up of any lumps in the mixture.

A potential application of the disclosed cell growth medium derived as a heated or dried biomass slurry is in cell cultured meat production. In an embodiment, the cell growth medium could be used as the biomass slurry (i.e. as a liquid stream) collected before the drying step (or prior to protein powder production). In this regard, the biomass slurry is exposed to hydrolyzing enzymes at this stage. The obtained hydrolysates are separated from the insoluble components (e.g. cell debris) and obtained liquid containing soluble components (protein hydrolysates, vitamins, minerals etc.) is dried into a powder keeping its solubility well preserved, and provided (such as by way of selling) to cell cultured meat producing companies.

In another embodiment, the cell growth medium could be used as the protein powder and provided to cell cultured meat producing companies. In this case, the protein powder is mixed with water to form the biomass slurry in a dispersed form. In this regard, 5-20% by weight of the protein powder is mixed in 80-95% by weight of sterile water until a homogenous mixture is achieved. The biomass slurry is treated by proteases, soluble components separated from the insoluble and used as cell growth medium in cell culturing. However, the biomass slurry obtained by mixing the protein powder and water might not be effectively hydrolyzed, thereby compromising the hydrolysis yield.

In yet another embodiment, the cell growth medium producing company and the cell cultured meat producing company could be co-existing in a location and the hydrolyzed biomass slurry could be directly fed to the cell cultured meat production in a continuous process. It will be appreciated that such co-existence of the two producers in a location would eliminate one drying step as the protein powder is anyway required to be hydrated before being used as a cell growth medium.

The present disclosure also relates to the system as described above. Various embodiments and variants disclosed above apply mutatis mutandis to the system.

Separation of liquid and solid phases of the biomass slurry could be performed in the separator, such as a centrifugal separator or a membrane filtration unit, widely used in industrial scales. The centrifugal separator typically uses centrifugation technique for separating components in a solution based on a particle size thereof. The membrane filtration unit operates based on filtration techniques that utilize a semi-permeable membrane to separate solid and liquid phases of a solution. It will be appreciated that centrifugal separators are preferred for concentrating bacterial cells as compared to filtration.

The reaction chamber is a special vessel for holding a volume of biomass slurry for treatment thereof by adding at least one protease thereto. It will be appreciated that the reaction chamber could receive a feed of the biomass slurry and a feed of the at least one protease continuously or in batches. Optionally, the reaction chamber is designed to withstand the conditions arising from enzymatic hydrolysis of the biomass slurry.

The incubator is a vessel for heating the biomass slurry. Notably, the temperature of the incubator is adjusted depending on the constituents of the biomass slurry, i.e. the cells, enzymes and so on. Besides temperature, the incubator also allows for regulation of other optimum conditions, such as humidity, air for example, at levels optimum for the cell growth. Optionally, an example of incubator is a water bath. It will be appreciated that using a water bath ensures heating while avoiding direct contact of the biomass slurry with heat. Optionally, the temperature in the incubator is from 80° C. up to 120° C.

The system further comprises a homogenizing device for homogenizing the biomass slurry with high pressure homogenization for at least partially degrading the microbial cells. The homogenizing device employs physical or mechanical ways of disrupting or homogenizing materials. Typically, used homogenizing devices include mortar and pestle, blenders, bead mills, sonicators, rotor-stator, and the like. The high-pressure homogenization (HPH) includes at least one pass, for example 1, 2, or 3, through the homogenizing device to increase cell disruption efficiency. Optionally, the homogenizing device is a high-pressure homogenizer and wherein the pressure in the high-pressure homogenizer is from 900 bars up to 2000 bars. The high-pressure homogenizers typically use very high pressures to disrupt the cell structures. Optionally, the homogenizing device is a liquid mill. The liquid mill typically uses shear forces to disrupt the cell structures.

Optionally, the system further comprises a heat-exchanger for incubating the biomass slurry. The heat-exchanger is a system used to transfer heat between two or more fluids. In this regard, the heat exchanger may have flow arrangement, parallel flow or counter flow, such that the heat-exchanging fluids travel in parallel to one another or in directions opposite to one another, respectively. Usually, heat-exchanger are widely used in industrial scales and are well known to a person skilled in the art. Optionally, the heat-exchanger is selected to be at least one of a tank heat-exchanger, a tubular heat-exchanger, or a plate heat-exchanger.

Optionally, the system further comprises a spray dryer for drying the microbial biomass slurry. The spray dryers enable rapidly drying the slurry material by using a hot gas. Spray drying is typically suitable for thermally-sensitive materials. Optionally, the spray dried product may be further milled or finished to a flake or powder form. Alternatively, optionally, the dryer is a drum dryer. The drum dryer is a rotating, high-capacity vessel configured to contain the slurry material, such as the biomass, and rotate the material therein at relatively low temperatures to produce sheets of drum-dried product.

Optionally, the at least one protease is selected to be at least one of a flavorzyme, an alcalase, a neutrase, a papain, a trypsin, a pepsin.

Optionally, the temperature in the reaction chamber for treating the biomass slurry by adding at least one protease is from 30° C. up to 75° C.

Optionally, a feed for gas fermentation in the bioreactor comprises at least one of selected from CO₂, CH₄, H₂, O₂, NH₃, at least one mineral.

Optionally, the system further comprises the microbial cells comprise an isolated bacterial strain VTT-E-193585 or a derivative thereof.

### EXPERIMENTAL PART

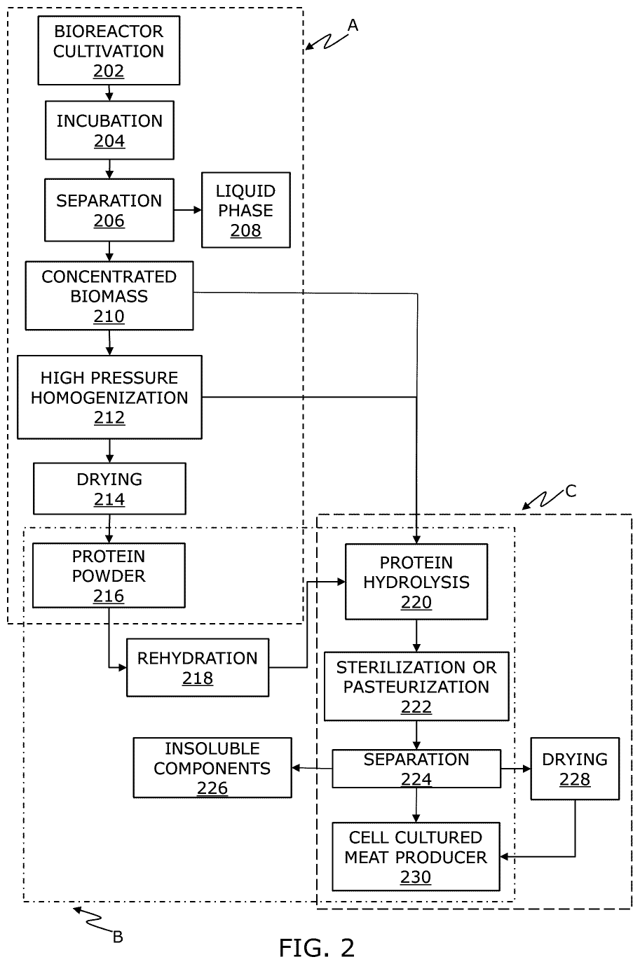

A cell growth medium was prepared according to steps of the present disclosure (FIG. 2). The analyses demonstrated that highest yield (by weight) and lowest average molecular weight of the cell growth medium determined with Size Exclusion Chromatography (SEC) was obtained by using a combination of Alcalase and Flavourzyme (Table 1). Flavourzyme alone gave the lowest yield.

TABLE 1

| | Enzyme | Yield (%) | Mw (g/mol) |
|---|---|---|---|
| Sample 1 | 2% Alcalase, 2% Flavourzyme | 40 | 482 |
| Sample 2 | 4% Alcalase | 36 | 654 |
| Sample 3 | 4% Flavourzyme | 22 | 506 |

### DETAILED DESCRIPTION OF THE DRAWINGS

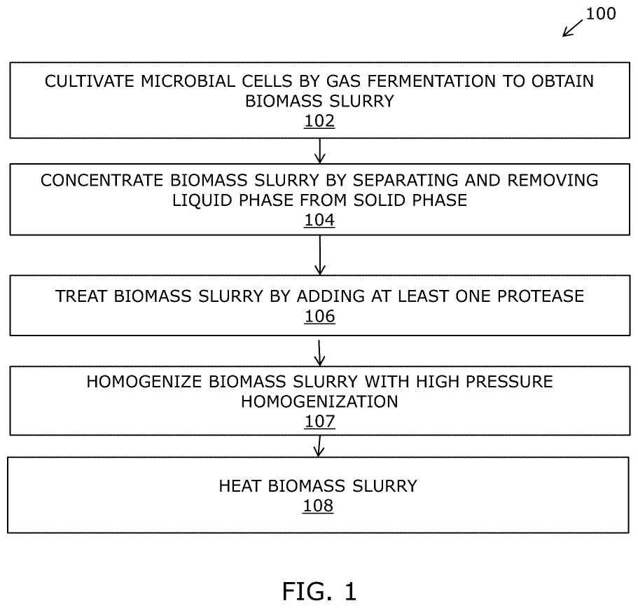

Referring to FIG. 1, there is shown a flowchart 100 illustrating steps of a method of producing a cell growth medium, in accordance with an embodiment of the present disclosure. At step 102, microbial cells are cultivated by gas fermentation to obtain a biomass slurry. At step 104, the biomass slurry is concentrated by separating and removing a liquid phase from a solid phase. At step 106, the biomass slurry is treated by adding at least one protease. At step 107, the biomass slurry is homogenized with high pressure homogenization. At step 108, the biomass slurry is heated.

The steps 102, 104, 106 and 108 are only illustrative and other alternatives can also be provided where one or more steps are added, one or more steps are removed, or one or more steps are provided in a different sequence without departing from the scope of the claims herein.

Referring to FIG. 2, depicted is a block diagram of a cell growth medium production line 200, in accordance with different embodiments of the present disclosure. As shown, cell growth medium production line 200 initiates with process A, when an inoculum of microbial cells is subjected to a bioreactor cultivation 202, using a cell culture medium and optimum conditions for growth, to form a biomass slurry (comprising a solid phase and a liquid phase 208). The cultivated biomass is subjected to incubation 204 by heat treatment. The incubated biomass is concentrated by separation 206 of the liquid phase 208 from a solid phase of the biomass slurry to yield a concentrated biomass 210. The concentrated biomass is subjected to a high-pressure homogenization 212 and subsequently, to drying 214 to yield a protein powder 216. At this stage, as shown in process B, the protein powder 216 is subjected to rehydration 226 and subsequently to protein hydrolysis 220, as shown in process C.

As shown in process C, the protein hydrolysis 220 is performed on the homogenized biomass slurry obtained from high-pressure homogenization 212 or alternatively directly from concentrated biomass. The hydrolyzed protein undergoes sterilization or pasteurization 222 to obtain a sterilized or pasteurized hydrolysate. Sterilization or pasteurization 222 is followed by separation 224, where the soluble components (proteins, peptides, amino acids, vitamins, minerals etc.) are separated from the insoluble components (cell debris etc.) and insoluble components 226 are removed to obtain a hydrolysate product that is provided, as cell growth medium, to a co-located cell cultured meat producer 230. An alternate step in the process B is subjecting the sterilized or pasteurized hydrolysate to a drying 228 to obtain a dried form of cell growth medium that is provided to a cell cultured meat producer 230, co-located or set-up distantly from the cell growth medium production line 200.

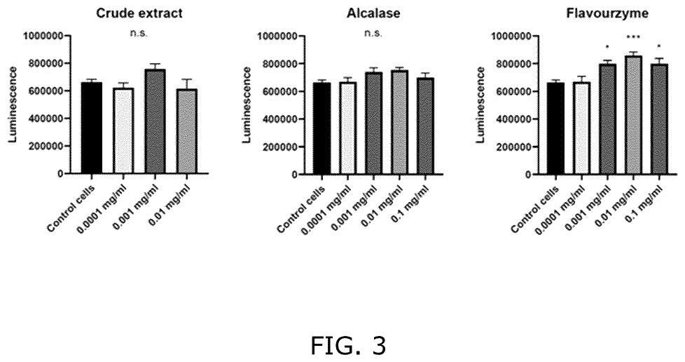

On FIG. 3, the bovine primary skeletal muscle cells were cultivated for 96 hours with supplementation of crude biomass slurry, Alcalase and Flavourzyme hydrolysates. The graphs show cell metabolic activity (adenosine triphosphate amount) in luminescence. Data represents mean±SEM (n=3 independent cell experiments seeded out in triplicates). (Asterix indicate significant differences, *<0.05, **<0.01, ***<0.001, ****p<0.0001. Abbreviations: n.s.—no significant difference).

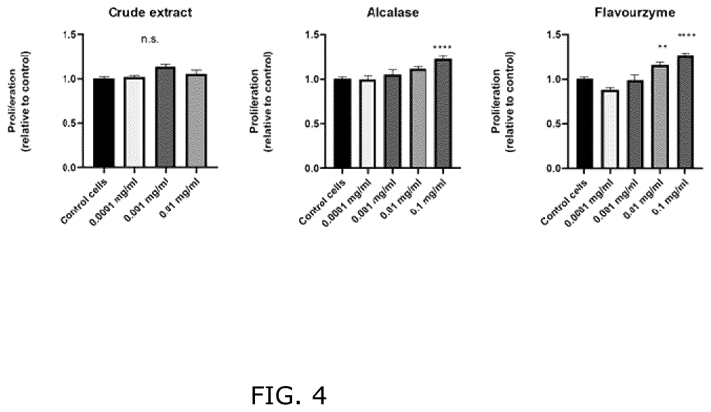

On FIG. 4, the bovine primary skeletal muscle cells were cultivated for 96 hours with supplementation of crude biomass slurry, Alcalase or Flavourzyme hydrolysates and the cells were analysed for DNA content. The graphs depict DNA normalised to control (ctr) cells. Data represents mean±SEM (n=3 independent cell experiments seeded out in triplicates). (Asterix indicate significant differences, *<0.05, **<0.01, ***<0.001, ****p<0.0001. Abbreviations: n.s.—no significant difference).

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A method of producing a cell growth medium, the method comprising
cultivating microbial cells by gas fermentation to obtain a biomass slurry;
concentrating the biomass slurry by separating and removing a liquid phase from a solid phase;
homogenizing the concentrated biomass slurry with high pressure homogenization, wherein the microbial cells are at least partially degrading;
treating the homogenized biomass slurry by adding at least one protease; and
heating the treated biomass slurry.

2. A method according to claim 1, further comprising separating and removing insoluble components from the heated biomass slurry.

3. A method according to claim 1, further comprising incubating the cultivated biomass slurry with a heat treatment.

4. A method according to claim 1, further comprising drying the heated biomass slurry.

5. A method according to claim 1, wherein the at least one protease is selected to be at least one of a flavorzyme, an alcalase, a neutrase, a papain, a trypsin, a pepsin.

6. A method according to claim 1, wherein heating the biomass slurry is carried out at temperature from 80° C. up to 130° C.

7. A method according to claim 1, further comprising adjusting the pH of the biomass slurry to be from 4.0 up to 10 before treating the biomass slurry by adding at least one protease.

8. A method according to claim 1, wherein treating the biomass slurry by adding at least one protease is carried out at temperature from 30° C. up to 75° C.

9. A method according to claim 1, further comprising
drying the concentrated biomass slurry to obtain a protein powder;
mixing the dried protein powder with water to obtain a biomass slurry.

10. A method according to claim 1, wherein a feed for cultivating by gas fermentation comprises at least one of selected from CO₂, CH₄, H₂, O₂, NH₃, minerals.

11. A method according to claim 1, wherein the microbial cells comprise an isolated bacterial strain VTT-E-193585 or a derivative thereof.

12. A system for producing cell growth medium, the system comprising:
a bioreactor for cultivating microbial cells by gas fermentation to obtain a biomass slurry;
a separator for separating a solid phase and a liquid phase of the biomass;
a homogenizing device for homogenizing the biomass slurry with high pressure homogenization for at least partially degrading the microbial cells;
a reaction chamber for treating the biomass slurry by adding at least one protease; and
an incubator for heating the biomass slurry.

13. A system according to claim 12 further comprising a separator to separate insoluble components from the heated biomass slurry.

14. A system according to claim 12, further comprising a heat-exchanger for incubating the biomass slurry.

15. A system according to claim 12, further comprising a spray dryer for drying the heated biomass slurry.

16. A system according to claim 12, wherein the at least one protease is selected to be at least one of a flavorzyme, an alcalase, a neutrase, a papain, a trypsin, a pepsin.

17. A system according to claim 12, wherein the temperature in the reaction chamber for treating the biomass slurry by adding at least one protease is from 30° C. up to 75° C.

18. A system according to claim 12, wherein a feed for gas fermentation in the bioreactor comprises at least one of selected from CO₂, CH₄, H₂, O₂, NH₃, at least one mineral.

19. A system according to claim 12, wherein the microbial cells comprise an isolated bacterial strain VTT-E-193585 or a derivative thereof.

20. A cell growth medium obtained by the method according to claim 1.
